(19) United States
(12) Patent Application Publication
(10) Pub. No.: US 2024/0198101 A1
(43) Pub. Date: Jun. 20, 2024

(54) SYSTEMS, APPARATUSES AND METHODS FOR PAIN MANAGEMENT

(71) Applicant: Gate Science, Inc., Moultonborough, NH (US)

(72) Inventor: Sanjay SINHA, Avon, CT (US)

(21) Appl. No.: 18/286,987

(22) PCT Filed: Apr. 13, 2022
(86) PCT No.: PCT/US2022/024686

§ 371 Date: Oct. 13, 2023

Related U.S. Application Data
(60) Provisional application No. 63/174,457, filed on Apr. 13, 2021.

(51) Int. Cl. A61N 1/36; A61M 5/142; A61M 25/00; A61N 1/05

(52) U.S. Cl. CPC A61N 1/36021; A61M 5/14244; A61M 25/0082; A61N 1/0551; A61M 2025/0095; A61M 2202/048; A61M 2205/04; A61M 2205/054; A61M 2210/1003; A61N 1/36034

(57) ABSTRACT

Embodiments of the present disclosure relate to, among other things, systems, devices, and related methods for operating the same, to treat or provide pain control via multimodal control of pain. Specifically, embodiments of the present disclosure are directed to by both the delivery of pulsed electrical current to and/or around a nerve trunk, and delivery of one or more substances to and/or around the nerve trunk.

### CROSS REFERENCE TO RELATED APPLICATIONS

This application claims priority to, and the benefit of, U.S. Provisional Application No. 63/174,457, filed on Apr. 13, 2021, the content of which is incorporated by reference in its entirety.

### FIELD OF THE DISCLOSURE

Disclosed herein are systems, devices and methods for providing pain control, more specifically, to pain control via neuromodulation using an electrically stimulating catheter, and/or, a therapeutic substance delivery catheter. In some embodiments, one or both catheters can be configured to deliver one or both of electrical pulses and a therapeutic fluid.

### BACKGROUND

Opioid abuse is a tragedy in society today—especially in the United States. The crisis is getting worse. In the last 20 years, there has been a 300% increase in annual opioid related deaths.

In 2019, 70,980 Americans died from drug overdose, up 4.6% from 2018. Moreover, the current pandemic fuels the epidemic, as drug deaths have risen an average of 13% in 2020; in fact, in May 2020, drug overdoses increased 42% year over year. Accordingly, on average, 128 people die each day from opioid overdose, and more than 10 million Americans misuse prescription opioids each year.

It has been determined that management of post-operative pain with opioids is a major factor driving abuse. Each year, more than 40 million Americans undergoing surgery are prescribed opioids for post-operative pain management. Each year, 2.6 million people who take opioids after surgery become persistent opioid users, and more than 600,000 will become addicted to opioids. Annual healthcare costs associated with addiction, approximately $14 billion, can be attributed to post-operative pain management.

The sensation of pain is transmitted from a region of the body by peripheral nerves to the dorsal horn of the spinal cord and then ascends up to the brain where it is interpreted as an unpleasant sensation of pain. The source of pain is usually tissue injury which activates the noci-receptors (inflammatory, trauma, thermal, chemical stimulus etc.). The noci-receptors pick up the information of pain and transmit it to the peripheral nerves. Pain is categorized as “acute pain” when it is generated as a result of obvious tissue injury or trauma (cuts, burn, broken bones, surgery etc.) from a region of the body. It is self-limited in nature lasting only days or weeks and completely resolves as the tissue heals. Once the pain resolves, the normal function returns.

When the pain persists beyond 12 weeks, the typical normal tissue healing time, it is defined as “chronic pain”. In some embodiments, acute pain after surgical trauma is very intense, severe pain that lasts for 24-72 hours. In some embodiments, thereafter, as healing occurs at the surgical site, the pain levels come down to moderate and mild levels and then completely resolve. In some embodiments, depending on the nature of the surgery, the entire episode of pain can last up to 4-6 weeks after surgery.

To control pain from a specific region of the body, the nerves innervating that specific region of the body is blocked with sodium channel blockade (molecular gate) or neuromodulated with electrical stimulation to block the synaptic gate. Depending on the region of the body, either a single nerve or multiple nerves need to be blocked to control the pain. A nerve block (molecular gate block) with sodium channel blockers is effective in controlling the acute, severe pain after surgery.

Electrical stimulation (neuromodulation) of peripheral nerves does not have the capacity to control severe pain experienced by a patient in the first 24-72 hours after surgery. When pain levels subside to moderate or mild levels, neuromodulation is effective in controlling the pain.

In a post-operative period, patients may develop transient episodes of severe pain termed as “breakthrough” pain; to this end, electrical stimulation alone is not sufficient to control these episodes of severe breakthrough pain, but sodium channel blockade will control the pain effectively.

In a case series of 11 patients having shoulder (rotator cuff) surgery, who received only electrical stimulation (neuromodulation) of brachial plexus for pain control, 64% (7/11) of the patients experienced severe intense pain in the recovery room and needed a rescue nerve block (molecular block) to control the pain. In addition, all patients required opioids, as neuromodulation was not enough to control the breakthrough pain (Ilfeld et al. Regional Anesthesia and Pain Medicine 2019; 44:310-318).

Similarly, in 7 patients having foot (bunionectomy) surgery, received only electrical stimulation of sciatic nerve for pain control. Rescue sciatic block was required in 3/7 (43%) patients in the recovery room. In addition, 3 patients required rescue block within 48 hours of surgery. Patients also needed at least one (median) opioid pill post-operatively during daily post-operatively (Ilfeld et al. Regional Anesthesia and Pain Medicine 2018; 43:580-589).

In 10 patients receiving only femoral nerve electrical stimulation for patients having knee surgery (Anterior Cruciate Ligament Repair), 5 (50%) patients requested a narcotic pill for pain in recovery room and 7 (70%) patients required a rescue adductor canal block in recovery room. In addition, in the first 2 days after surgery, 8 patients (809%) need a rescue adductor canal block. On day 3 after surgery, 40% continued to require rescue adductor canal block. (Ilfeld et al. Neuromodulation 2019; 22:621-629).

Thus, there is an urgent clinical need for effective management of post-operative pain in hospital and ambulatory surgical centers. To this end, molecular block of sodium channel and blockade of synaptic gate in the dorsal horn of spinal by electrical stimulation nerve can be controlled along the nerve trunk. Molecular blockade is effective for early, intense post-surgical or post-traumatic pain lasting for hours and break-through pain, but has the potential impair motor function. A synaptic blockade is effective for moderate pain lasting for weeks and does not impair motor function.

### SUMMARY

Embodiments of the present disclosure relate to, among other things, systems, devices, and related methods for operating the same, to treat or provide pain control via multimodal control of pain. Specifically, embodiments of the present disclosure are directed to provide both the delivery of pulsed electrical current to and/or around a nerve trunk, and delivery of one or more substances to and/or around the nerve trunk.

In some embodiments, such a system includes two catheters independently positioned along the nerve trunk, where one is configured to deliver a sodium channel blocker medication from an exit port, closing the molecular gate on the nerve trunk, and the other is configured to deliver pulsed electrical stimulation to disrupt summation of synaptic activity in the dorsal horn with a stimulating electrode, closing the synaptic gate.

In some embodiments, a single catheter placed on a nerve trunk is initially used for injection of sodium channel blocker medication and when the effects of the medication has dissipated, the same catheter can then be used for delivery of electrical current to block the synaptic gate at the dorsal horn.

In some embodiments, a pulse generator is provided for synaptic blockade and a bolus pump is provided for molecular blockade—which (in some embodiments) can be clipped together into a single unit or clipped on a belt individually as a wearable device, which may include detachable leads and tubing (e.g., for bathing). In some embodiments, the system can include control mechanisms for the patient to adjust neuromodulation (e.g., a dial to control the amplitude of a pulsed electrical signal for up to a predetermined period of time (e.g., 30 days). Breakthrough pain, in some embodiments, can be treated via small injection of anesthetic initiated by patient (via the pump/catheter). Thus, a patient can stop pain with the touch of a button.

Accordingly, in some embodiments, a pain management or treatment method is provided which includes implanting a first catheter at a first location such that a distal end of the first catheter is proximate a nerve trunk of a patient, and implanting a second catheter at a second location such that a distal end of the second catheter is proximate the nerve trunk and spaced away from the distal end of the first catheter. At least one of the first catheter and the second catheter include at least one electrode configured to provide a pulsed electrical current to and/or around the nerve trunk, and at least one of the first catheter and the second catheter are configured to deliver an amount of a substance to and/or around the nerve trunk.

Such embodiments may include one and/or another of (in some embodiments, a plurality of, in some embodiments, a majority of, and in some embodiments, substantially all of or all of) the following steps, features, functionality, structure, and clarifications, yielding yet further embodiments of the present disclosure:
both of the first catheter and the second catheter include at least one electrode or more configured to provide the pulsed electrical current to and/or around the nerve trunk;
both of the first catheter and the second catheter are configured to deliver an anesthetic to and/or around the nerve trunk;
the first catheter includes the at least one electrode or more configured to provide the pulsed electrical current to and/or around the nerve trunk, and the second catheter is configured to deliver an anesthetic to and/or around the nerve trunk;
only the first catheter includes at least one electrode or more configured to provide the pulsed electrical current to and/or around the nerve trunk, and only the second catheter is configured to deliver an anesthetic to and/or around the nerve trunk;
the at least one electrode comprises at least a pair of electrodes;
at least one of the first catheter and the second catheter includes a removable needle;
at least one of the first catheter and the second catheter includes a removable needle having a lumen,
the catheter that is configured with the at least one electrode:
   the at least one electrode comprises at least a pair of electrodes;
   at least one of the first catheter and the second catheter includes a removable needle having a lumen, and
   one of the electrodes of the electrode pair comprises the distal end of the needle;
the delivery of the amount of the substance is configured to close the molecular gate (sodium channel or sodium-potassium pump) in the axonal membrane of the nerve trunk;
the substance comprises at least one of a sodium channel blockers (anesthetic agent) and/or an adjuvant added to anesthetic agent;
the amount of at least one of the sodium channel blocker and the anesthetic amount of the anesthetic is between about 0.1 mL and 30 ml;
the delivery of the pulsed electrical current delivered to the nerve trunk is configured to close the synaptic gate in the dorsal horn of the spinal cord/dorsal root ganglion;
the pulsed electrical current comprises a current of between 0.1 and 30 milliamps (and ranges there between);
the pulsed electrical current includes pulses of between 0.1 and 1.0 millisecond (and ranges there between);
the second location is spaced away from the first location by between 1 and 30 cm (and ranges therebetween);
the nerve trunk contains the nerve pathway for a body part of the patient;
the body part is selected from the group consisting of: the neck, the shoulder, the elbow, the hand, the wrist, the spine (cervical, thoracic, lumbar, sacral), the hip, the thigh, the knee, the leg, the ankle and the foot, chest wall, abdominal wall and perineum;
the method is performed for pain arising from an area of the body from a surgical procedure, trauma and or chronic pain;
the pulsed electrical current is supplied via a pulse generator;
the amount of medication is supplied via a pump;
at least one of the first catheter and second catheter are implanted under ultrasound guidance or x-ray guidance;
applying a first electrically conductive skin patch on the skin of the patient, the first skin patch is configured to complete an electrical circuit with a/the pulse generator and distal end of the needle housed inside the catheter with only the needle tip protruding from the distal end of the catheter and serving as a unipolar electrode
delivering the substance in and/or around the nerve trunk via the at least one of the first catheter and second catheter,
   the first substance can be delivered for a first time period;
and/or
delivering the pulsed electrical current in and/or around the nerve trunk via the at least one of the first catheter and second catheter having the at least one electrode;
   the pulsed electrical current can be delivered for second time period;
   the pulsed electrical current can be delivered after delivery of a/the substance by at least one of the first catheter and the second catheter;
   and/or
   the pulsed electrical current can be delivered a period of time after delivery of the substance by at least one of the first catheter and the second catheter;

In some embodiments, a pain management/treatment system or kit is provided and includes a first catheter configured for implantation at a first location such that a distal end of the first catheter is proximate on a nerve trunk of a patient, and a second catheter configured for implantation at a second location such that a distal end of the second catheter is proximate the nerve trunk and spaced away from the distal end of the first catheter. At least one of the first catheter and the second catheter include at least one or more electrodes configured to provide a pulsed electrical current to and/or around the nerve trunk, and at least one of the first catheter and the second catheter are configured to deliver an amount of a substance (medication) to and/or around the nerve trunk.

Such embodiments may include one and/or another of (in some embodiments, a plurality of, in some embodiments, a majority of, and in some embodiments, substantially all of or all of) the following steps, features, functionality, structure, and clarifications, yielding yet further embodiments of the present disclosure:
both of the first catheter and the second catheter include at least one or more electrodes configured to provide the pulsed electrical current to and/or around the nerve trunk;
both of the first catheter and the second catheter are configured to deliver an anesthetic medication to and/or around the nerve trunk;
the first catheter includes the at least one or more electrodes configured to provide the pulsed electrical current to and/or around the nerve trunk, and the second catheter is configured to deliver the substance to and/or around the nerve trunk;
only the first catheter includes the at least one or more electrodes configured to provide the pulsed electrical current to and/or around the nerve trunk, and only the second catheter is configured to deliver an anesthetic to and/or around the nerve trunk;
the first catheter configured to deliver electrical current is positioned proximal on the nerve trunk (closer to the dorsal horn of spinal cord) while the second catheter is positioned distal to the first catheter (farther away from the spinal cord compared to first catheter)
the at least one electrode comprises at least a pair of electrodes;
at least one of the first catheter and the second catheter includes a removable needle having a lumen,
   the at least one electrode can comprise at least a pair of electrodes;
   at least one of the first catheter and the second catheter can include a removable needle having a lumen, and
   one of the electrodes of the electrode pair can comprise the distal end of the needle;
a vial of the substance;
the substance can comprise at least one of a sodium channel blocker and an adjuvant medication;
a pulse generator for supplying the pulsed electrical current (and/or for control of electrical signal amplitude);
a pump for supplying the substance to the at least one of the first catheter and second catheter;
a garment wearable by the patient for housing at least one of a/the pulse generator and a/the pump;
at least one of the following components:
   at least one electrical cord for at least one of the first catheter and the second catheter;
   at least one needle for at least one of the first catheter and the second catheter;
   at least one tube-set for at least one of the first catheter and the second catheter for connection thereof with a/the pump for delivering the substance;
   at least one filter configured to be in fluid communication with at least one of the first catheter and the second catheter, the filter to configured to filter the substance prior to the substance being delivered to the nerve trunk;
   at least one dressing configured to retain at least one of the first catheter and the second catheter in place while implanted into tissue of the patient;
   at least one electrical cord configured to transmit (conduct) electrical current between a/the pulse generator and at least one of the first catheter and the second catheter;
   at least one EKG patch;
   at least one EKG patch cord;
   at least one biopatch configured to receive pulsed electrical current from the patient via the skin surface;
   at least two hub management system to affix the hubs to the skin and organize the electrical wire and drug tubing;
   and
   at least one syringe;
the system/kit can include two or more of the above listed components;
the system/kit comprises a majority of the above listed components;
the system/kit comprises all of the above listed components.

The above noted embodiments, as well as objects and advantages thereof, will become even more evident by the following detailed description, and corresponding drawings, a brief description of which is set out below.

### BRIEF DESCRIPTION OF THE DRAWINGS

FIG. 1 schematically illustrates an overview of a system for pain management, according to some embodiments;

FIG. 2 illustrates an overview of components of a system for pain management, for use in, for example, a kit, according to some embodiments;

FIG. 2A illustrates an overview of a catheter delivery apparatus for effecting neuromodulation, and/or therapeutic fluid delivery, into tissue, according to some embodiments;

FIG. 2B illustrates an enlarged view of a distal end of the apparatus of FIG. 2A;

FIG. 2C illustrates a view of the catheter portion of the apparatus of FIG. 2A, according to some embodiments;

FIG. 2D is a view of the catheter portion of the apparatus of FIG. 2A, in combination with a filtering device;

FIG. 3A is an overview of the operation of a neuromodulation aspect of the disclosure according to some embodiments;

FIG. 3B is an overview of the operation of a neuromodulation aspect according to some embodiments,

FIG. 3C illustrates an overview of a system for pain management, according to some embodiments,

FIG. 4 illustrates an overview of a wearable garment for other components of the system for pain management, as shown in FIG. 3, according to some embodiments;

FIGS. 5A-B are human anatomy illustrations of a human shoulder, illustrating nerve, muscle and tissue (among other tissue), including one or more nerve trunks; FIG. 5B illustrating placement of pain management components of a system for pain management of shoulder (e.g., a catheter for supplying pulsed electrical current, and a catheter for delivering a substance to the nerve trunk) according to some embodiments;

FIG. 6 is a human anatomy illustration of groin and upper thigh, illustrating nerve, muscle and bone tissue (among other tissue), including one or more nerve trunks, illustrating placement of pain management components of a system for pain management of knee (e.g., a catheter for supplying pulsed electrical current, and a catheter for delivering a substance to the nerve trunk) according to some embodiments.

FIG. 7A is a representation of the brachial plexus nerve system which is made up of roots, trunks, divisions, cords and terminal branches.

FIG. 7B is a representation of the lumbar plexus and the sacral plexus nerve systems including the femoral nerves, obturator nerve and the lateral femoral cutaneous nerve.

FIG. 8A shows current flowing from the electrical pulse generator to the distal electrode on the catheter; the current travels in the tissue and returns to the electrical pulse generator via skin patch.

FIG. 8B shows current flowing from the external pulse generator to the proximal electrode on the catheter; the current flows through the tissue and returns back to the external pulse generator via skin patch.

FIG. 8C shows current of the same polarity flowing to the electrode pair on the catheter and the current returns back to the external pulse generator via skin patch.

Accordingly, theses drawings, which are incorporated in and constitute a part of this specification, illustrate some of the exemplary embodiments of the present disclosure and together with the description, serve to explain specific principles of at least some embodiments of the disclosure.

### DETAILED DESCRIPTION

While the present disclosure is described herein with reference to illustrative embodiments for particular applications, it should be understood that embodiments of the present disclosure are not limited thereto. Other embodiments are possible, and modifications can be made to the described embodiments within the spirit and scope of the teachings herein, as they may be applied to the above-noted field of the present disclosure or to any additional fields in which such embodiments would be of significant utility.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to), according to the respective embodiment(s) noted.

Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As earlier noted, embodiments of the present disclosure relate to, among other things, systems, devices, and related methods for operating the same, to manage, treat or provide pain control via multimodal control. Specifically, embodiments of the present disclosure are directed to use of components, devices and systems, as well as corresponding methods, to manage pain by delivery of pulsed electrical current to and/or around a nerve trunk, and in addition, delivery of one or more substances to and/or around the nerve trunk. In some embodiments, such components, devices and systems are used according to disclosed methodology.

This patent application hereby incorporates several applications by reference, including PCT application no. PCT/US2020/024435, having an international filing date of Mar. 24, 2020, as well as United States Provisional Patent Application Serial Nos. 62/123,332, titled “Neuromodulation Catheter,” filed on Mar. 23, 2019, and 62/911,001, titled “Multi-Lumen, Multi-Electrode Neuromodulation Cather,” filed on Oct. 4, 2019. Each of the foregoing disclosures are incorporated by reference herein in their entireties. The embodiments, and any of the features and functionality thereof, can be combined with features, and/or functionality of the embodiments of the present disclosure, yielding yet further embodiments of the present disclosure.

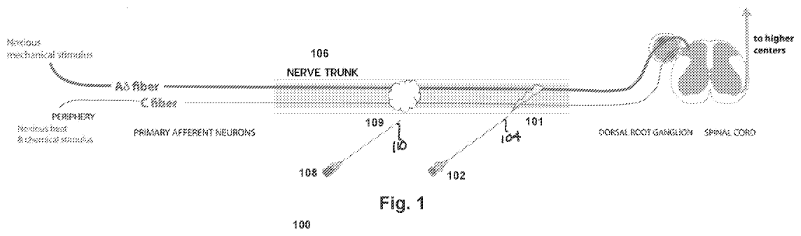

Accordingly, in some embodiments, as shown in FIG. 1, a pain management or treatment method is provided which includes implanting a first catheter 102 of a pain management system 100 at a first location 101 with respect to tissue or area of the body of a patient. The first location is such that a distal end 104 of the first catheter 102 is proximate a nerve trunk 106 of the patient. In some embodiments, the method includes implanting a second catheter 108 at a second location 109 such that a distal end 110 of the second catheter 108 is proximate the nerve trunk 106 and is spaced away from the distal end 104 of the first catheter 102. At least one of the first catheter 102 and the second catheter 108 include at least one or more electrodes 110 (see FIG. 1B) configured to provide a pulsed electrical current to and/or around the nerve trunk 106, and at least one of the first catheter 102 and the second catheter 108 are configured to deliver an amount of a substance to and/or around the nerve trunk.

Such embodiments may include one and/or another of (in some embodiments, a plurality of, in some embodiments, a majority of, and in some embodiments, all of) the following steps, features, functionality, structure, and clarifications, yielding yet further embodiments of the present disclosure.

For example, both of the first catheter and the second catheter can include the at least one or more electrodes 112 configured to provide the pulsed electrical current to and/or around the nerve trunk. In some embodiments, both of the first catheter 102 and the second catheter 108 can be configured to deliver an anesthetic to and/or around the nerve trunk.

In some embodiments, the first catheter 102 includes at least one or more electrodes 112, for providing the pulsed electrical current to and/or around the nerve trunk, and the second catheter 108 is configured to deliver an anesthetic to and/or around the nerve trunk. In still other embodiments, only the first catheter 102 includes the at least one or more electrodes 112 (for providing the pulsed electrical current to and/or around the nerve trunk), and only the second catheter 108 is configured to deliver an anesthetic to and/or around the nerve trunk.

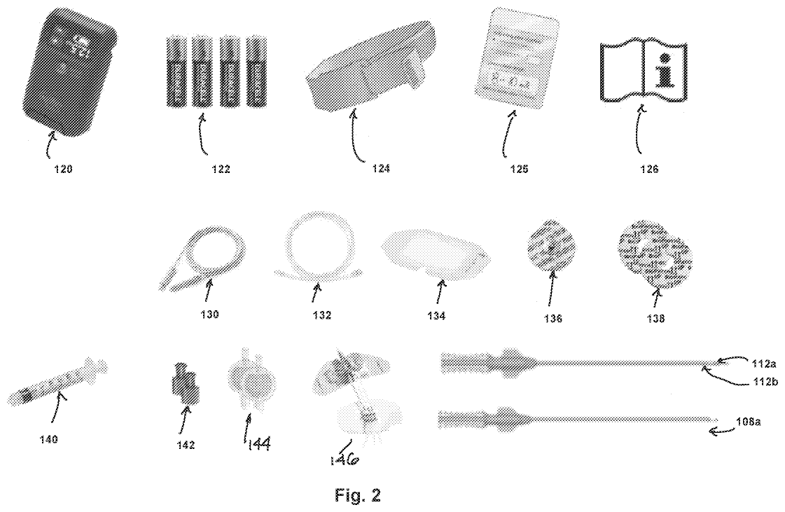

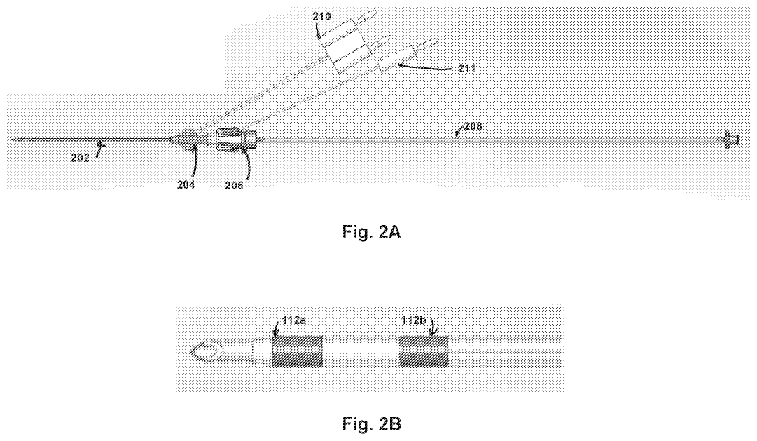

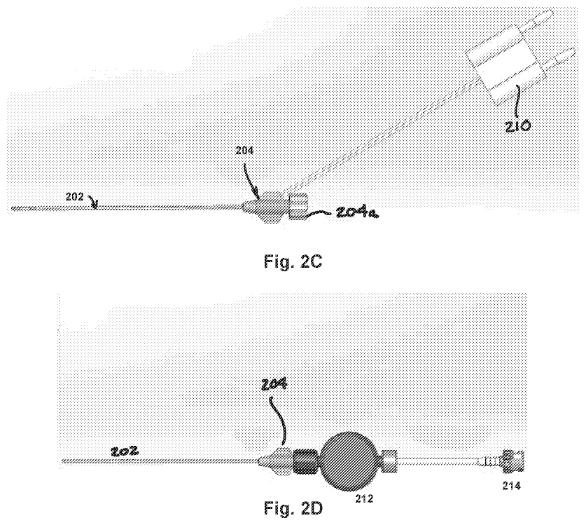

In some embodiments, and as shown in FIG. 2B, the at least one electrode comprises at least a pair of electrodes 112a and 112b and are configured for electrical connection to a pulse generator. In some embodiments, at least one of the first catheter 102 and the second catheter 108 includes a removable needle 114. At least one of the first catheter 102 and the second catheter 108 includes a removable needle 114 having a lumen 116, and a distal end 118.

In some embodiments, the at least one electrode comprises at least a pair of electrodes, where at least one of the first catheter 102 and the second catheter 108 includes a removable needle 114 having a lumen 116 and one of the electrodes of the electrode pair comprises the distal end 118 of the needle.

In some embodiments, the delivery of an amount of the substance is configured to close the molecular gate of the sodium channel in the axonal membrane of the nerve trunk, where the substance can comprise at least one of a sodium channel blocker and an adjuvant. In some embodiments, the amount of the at least one of the sodium channel blocker and/or the adjuvant is between about 0.5 and 30 ml (and ranges therebetween). With respect to pulsed electrical current delivery, the delivery of the pulsed electrical current is configured to close the synaptic gate of the nerve trunk, and in some embodiments, the pulsed electrical current comprises a current of between 0.1 and 30 milliamps (and ranges therebetween), and can includes pulses of between 0.01 and 1 millisecond.

In some embodiments, the second location is spaced away from the first location by between 10 and 100 mm (and ranges therebetween) but depends upon the nerve trunk location and arrangement within tissue. Thus, for example, to manage pain with respect to surgical repair of the rotator-cuff, the catheter are arranged to address pain by placement proximate the brachial plexus nerve in the interscalene space, which blocks pain with respect to the first part of the brachial plexus, where the nerves to the shoulder come from. In such a procedure, the first catheter delivering electrical pulses is spaced apart from the catheter delivering a therapeutic substance between, in some embodiments, 10 mm and 80 mm (and ranges therebetween). Accordingly, system, devices and methods according to some of the embodiments of the present disclosure can be used to manage pain for surgical procedures and injuries for many body arears/parts/tissue including, for example those of the shoulder, the hand, the wrist, the spine, the hip, the knee, the ankle and the foot.

FIG. 2 illustrates components for a pain management system according to some embodiments, of which each may be distributed as is, and in some embodiments, combined with one and/or another component as a kit for distribution, and in some embodiments, a majority of the components can be assembled as a kit distribution, and in some embodiments, all of the components can be assembled as a kit for distribution. To this end, the components, according to some embodiments can include a pulse generator device (PGD) 120, batteries 122, an PGD carrier 124, reference material 125, and instructions for use 126. These components, can be a part of a non-sterile assemblage, configured in non-sterile packaging.

The system may, in some embodiments, also include a therapeutic fluid delivery device (e.g., bolus pump which may also be referred to as a pump) 128, one or more electrical connection cords 130, one or more lengths (which may include corresponding connectors) therapeutic fluid delivering tubing 132, one or more dressings 134, one or more EKG patches 136 (which may include associated cords), one or more (in some embodiments two) bioelectric patches 138, one or more syringes 140, one or more various caps or connectors 142 for various components (e.g., tubing, cords), one or more filters (in some embodiments, two) 144, one or more hubs (e.g., for fluid and/or electrical connection(s)) 146, one or more catheters (e.g., 102a) which, in some embodiments, includes one or more, and preferably, a plurality of electrodes 112a, b at, e.g., a distal end thereof (e.g., two electrodes), and one or more bolus catheters (e.g., 108a), which in some embodiments, may also include one or more electrodes. In some embodiments, the catheters can include a length of between 6 cm and 20 cm, and ranges therebetween (in some embodiments, 6 cm or 20 cm). These components, can be a part of a sterile assemblage, configured in non-sterile packaging.

Regarding the kit embodiments noted above, any therapeutic fluid (e.g., anesthetic drug) that would be dispensed, of course, is provided separately (though, if legal, can be packaged with any of the kits noted above). Additionally, the combination of non-sterile and sterile components/packaging can comprise a kit. Such a kit can correspond to a bolus pump and neurostimulation system. However, in some embodiments, the pump 128 and bolus pump and catheter components can be eliminated, and such a kit can correspond to a neurostimulation system/kit for delivery of electrical pulses only.

In some embodiments, catheters include a short (e.g., 6 cm), and a long catheter (e.g., 20 cm), at least one of which, and in some embodiments, both of which includes needles (which can be configured for removable insertion within the catheter). Accordingly, in some embodiments, the catheter can comprise a 17 gauge catheter which is associated with a 19 gauge needle. Spacing of electrodes on one and/or another of the catheters can include the same spacing (e.g., arranged at the distal end of a respective catheter).

FIG. 2A illustrates an overview of an apparatus for effecting neuromodulation and therapeutic fluid delivery into tissue, according to some embodiments. As shown, the apparatus includes a catheter 202, a catheter hub 204, a needle hub 206 (which includes a corresponding needle for insertion within the catheter hub and catheter 202. The apparatus can also include a syringe tubing 208 removably connectable therewith (e.g., for hydrodissection). The apparatus may also include a catheter electrical connector 210, and a needle electrical connector 211, configured for electrical connection to the, for example, to the PGD (and/or other device), which can supply current/pulses to the electrodes arranged on the catheter 202 and/or a needle (contained within the catheter). Such a connector can include a single electrical connection, or, more than one (e.g., 2, 3, 4 or more) so as to provide electrical communication between the electrodes on the catheter and/or between the needle and the NPGD (and/or other device). FIG. 2B is described above.

FIG. 2C illustrates a view of the catheter portion of the apparatus of FIG. 2A, according to some embodiments, illustrating the catheter 202, the catheter hub 204, and a catheter cap 204a, which is configured for connection with the catheter hub to seal it (when the catheter hub is not connected to other tubing). Also illustrated is an electrical connector 210 which is used, in some embodiments, to communicate electrical pulses/current from the NPGD to the electrode(s) of the catheter and/or a needle.

FIG. 2D is a view of the catheter portion of the apparatus of FIG. 2A, which illustrates the catheter in combination with a filtering device 212 (e.g., 0.2 micron filter), as well as a tube/tubing 214 (or quick-disconnect tube-set) for connecting to and/or supplying, for example, a therapeutic fluid to the needle and/or catheter. The filter may be configured to block particles of a certain desired size, e.g., in some embodiments, blocking particles greater than 0.2 microns. As also shown are catheter 202, and catheter hub 204.

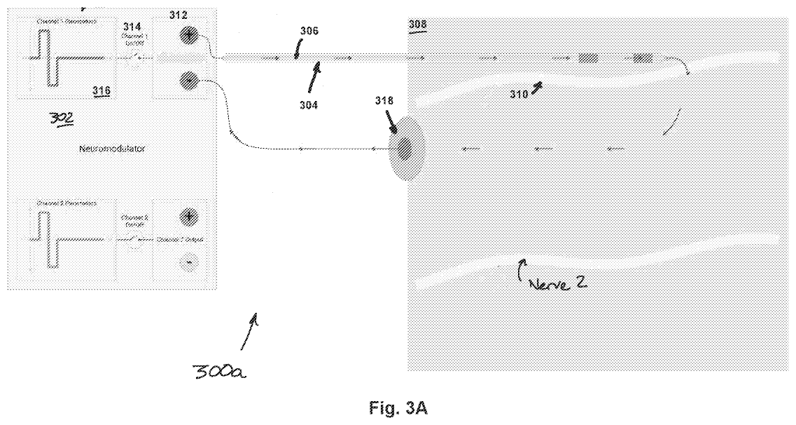

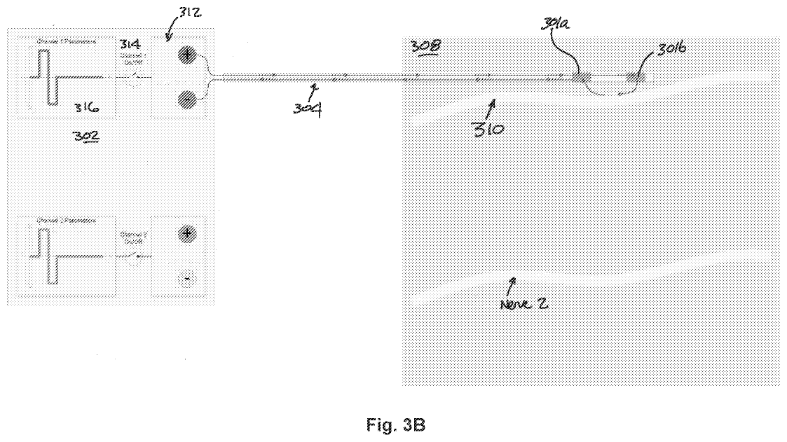

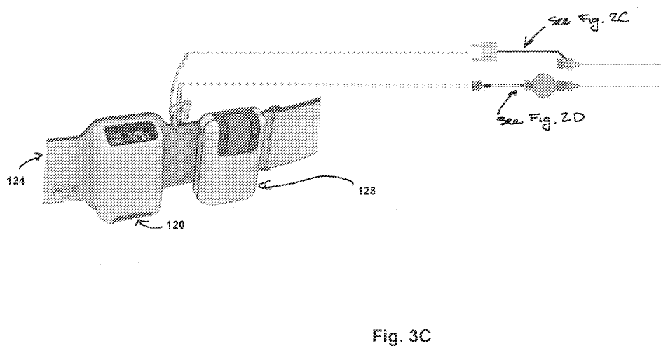

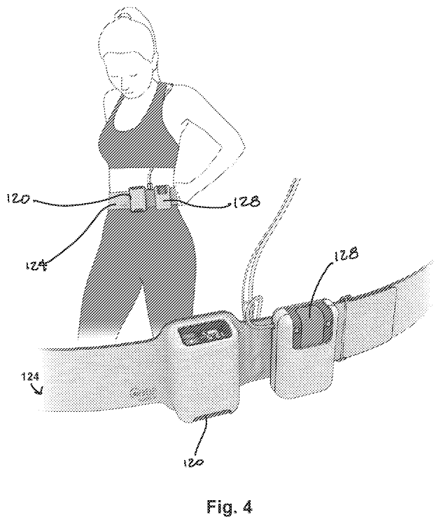

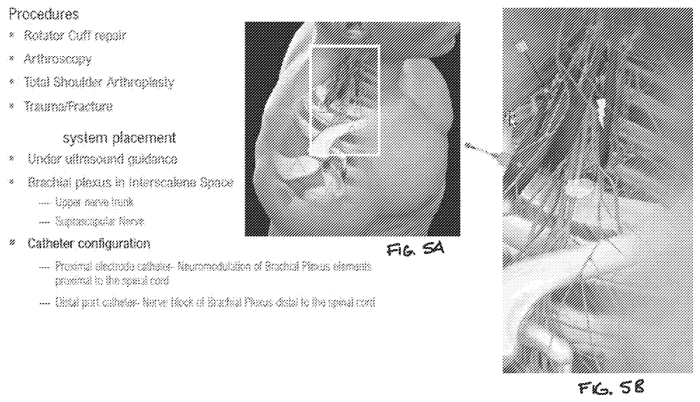

FIGS. 3A-B illustrate an overviews of operation of the neuromodulation aspect of some of the embodiments of the present disclosure. As shown in FIG. 1, the neuromodulation aspect is used in conjunction with the therapeutic substance delivery aspect for a total system and method of pain management.

Accordingly, FIG. 3A is an overview of the operation of a neuromodulation system 300a according to some embodiments, illustrating locating/arranging a first neuromodulation apparatus 302 in and among (depending upon the component) in tissue, as well as electrical connections and electrical current/pulse flow. Accordingly, in some embodiments, a first catheter 304 with a first needle 306 inserted therein which, in some embodiments, is configured to be inserted into tissue of a patient (e.g., through the skin 308), such that, the distal end of the first catheter 304 and first needle 306 are proximate a first nerve 310 to be stimulated by electrical current for placing catheter in close proximity to the nerve. In such embodiments, the needle 306, which is configured to conduct electric (in some embodiments), is in electrical connection/communication with a positive (for example) terminal 312 of a first channel 314 of a PGD 316. A skin patch 318 configured to communicate electrical pulses/current in/from skin/tissue (e.g., see EKG patch FIG. 1), is arranged on the skin 308 of the patient, preferably, in some embodiments, proximate the first nerve 310, and is in electrical connection/communication with a negative (for example) terminal 320 of the first channel 314 of the PGD, such that, the catheter/needle, tissue, and a skin patch form a closed circuit. Accordingly, activation of the PGD 316 for electrical stimulation on the first nerve 310 directs electrical pulses from the positive terminal of the first channel 314, down the needle 306 to the distal end thereof, such that, the pulses pass proximate to and/or through the first nerve 310 (the electrical path illustrated by the arrows from the first terminal 312, to the needle 310, to the nerve/tissue, to the skin patch 318 and back to the PGD 316 via the second terminal.

Each channel (or a channel) of the PGD can be configured to supply electrical pulses according to, in some embodiments, a predetermined voltage, current, and pulse time, and for a predetermined period of time. A nerve stimulator used for connecting with the needle for purpose of stimulating a target nerve for placing a catheter in close proximity to the nerve will have settings of 0.1-5.0 milliamps (and ranges therebetween), pulse duration of 0.1 milliseconds and frequency of 2-4 Hertz (and ranges therebetween). With these setting, as the electrified “active” needle tip approaches a target nerve, muscle groups innervated by the nerve will begin to twitch. If a muscle twitch can be elicited at a current of 0.5-0.8 milliamps (and ranges therebetween), the catheter tip is close proximity to the nerve and will result in successful nerve block by injection of sodium channel blockade medication and/or neuromodulation be electrical stimulation

FIG. 3B is an overview of the operation of a neuromodulation system 300b according to some embodiments, in which the first catheter 304 lacks a needle therein, and the systems lacks a/the skin patch 318. Like the embodiments illustrated in FIG. 3A, placement of the catheter is such that at least the distal end thereof is arranged proximate the first nerve. The catheter 304, in the illustrated embodiment, includes two electrodes 301a-b (e.g., a plurality) on the distal end thereof, with each being in communication with one of the terminals 312 of a (e.g., first) channel 314 of the PGD 316, forming a complete circuit. Accordingly, activation of the PGD for neuromodulation on the first nerve directs electrical pulses from the positive terminal of the first channel, down to a first of the electrodes at the distal end of the catheter, exiting the first electrode so as to direct electrical pulses/current in tissue proximate the first nerve (as the pulses/current follow the electrical circuit and returning to the PGD via the second electrode back to the second terminal of the first channel. The current/pulse flow is illustrated by the arrows included in the figure.

In some embodiments, a pain management or treatment system or kit is provided (see, e.g., FIGS. 1, 2, 2A-D, 3C and 4) and includes a first catheter 102 configured for implantation at a first location such that a distal end 104 of the first catheter 102 is proximate a nerve trunk of a patient, and a second catheter 108 configured for implantation at a second location on the same nerve trunk such that a distal end 110 of the second catheter 108 is proximate the nerve trunk and spaced away from the distal end 104 of the first catheter 102. At least one of the first catheter and the second catheter include at least one electrode configured to provide a pulsed electrical current to and/or around the nerve trunk, and at least one of the first catheter and the second catheter are configured to deliver an amount of a substance to and/or around the nerve trunk.

Additionally, in some embodiments, at least one of the first catheter and the second catheter can include a removable needle having a lumen, the at least one electrode can comprise at least a pair of electrodes. Moreover, in some embodiments, the kit can include one or more of:
a vial or syringe 140 of the therapeutic substance;
a pulse generator 120 for supplying the pulsed electrical current;
a pump 128 for supplying the substance to the at least one of the first catheter and second catheter;
a garment 124 wearable by the patient for housing at least one of a/the pulse generator and a/the pump;
and/or
at least one of the following components (in some embodiments, a plurality of the following, in some embodiments, a majority of the following, and if not mutually exclusive, substantially all or all of the following):
   at least one needle (306) for at least one of the first catheter and the second catheter;
   at least one tube-set 132 for at least one of the first catheter and the second catheter for connection thereof with a/the pump for delivering the substance;
   at least one filter 144 configured to be in fluid communication with at least one of the first catheter and the second catheter, the filter to configured to filter the substance prior to the substance being delivered to the nerve trunk;
   at least one dressing 134 configured to retain at least one of the first catheter and the second catheter in place while implanted into tissue of the patient;
   at least one electrical cord 130 configured to communication electrical current between a/the pulse generator and at least one of the first catheter and the second catheter;
   at least one EKG patch 136, configured to receive pulsed electrical current from the patient via the skin surface;
   at least one EKG patch cord;
   at least one biopatch 138 to be placed at the skin insertion site of catheter to prevent infection;
   at least one hub 146;
   and
   at least one syringe 140;
the kit can include two or more of the above listed components;
the kit comprises a majority of the above listed components;
and
the kit comprises all of the above listed components.

In some embodiments, two or more components of the disclosed neuromodulation apparatuses and systems can be combined to form a single unit. For example, the pump may be incorporated into the design of the pulse generator so that the combined device may deliver the electrical impulses for neuromodulation and delivery of a/the therapeutic fluid. In some embodiments, the pump and/or pulse generator may be programmable. For example, the pump may be programmed to deliver a continuous infusion of fluid or a bolus thereof, or a combination of both. A computer, one or more processors, and one or more interfacing units may be used to control the pump's programming. In some embodiments, both neuromodulation and delivery of the therapeutic fluid can be performed for a limited time period (e.g., 2-6 days, 3-5 days, and ranges therebetween), then, neuromodulation only of the nerve/tissue thereafter. In some embodiments, only neuromodulation is performed.

FIGS. 5A-B are human anatomy illustrations of a human shoulder, illustrating nerve, muscle and tissue (among other tissue), including one or more nerve trunks; where FIG. 5B illustrates placement of pain management components of a system for pain management (e.g., a catheter for supplying pulsed electrical current in the interscalene space, and a catheter for delivering a substance (medication) to the distal branches of the brachial plexus e.g. upper trunk of brachial plexus or suprascapular nerve) according to some embodiments.

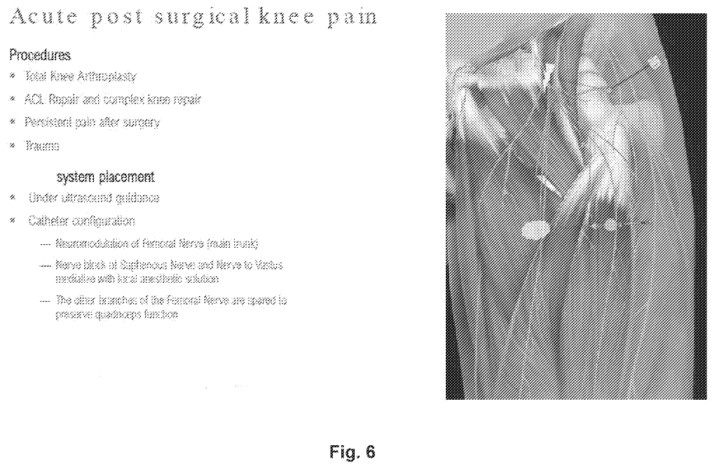

Similarly, FIG. 6 is a human anatomy illustration of a human groin, illustrating the femoral nerve, muscle and bone tissue (among other tissue), including one or more branches of the femoral nerve, illustrating placement of pain management components of a system for pain management of knee (e.g., a catheter for supplying pulsed electrical current placed at femoral nerve, and a catheter for delivering a substance to the nerves in the adductor canal) according to some embodiments.

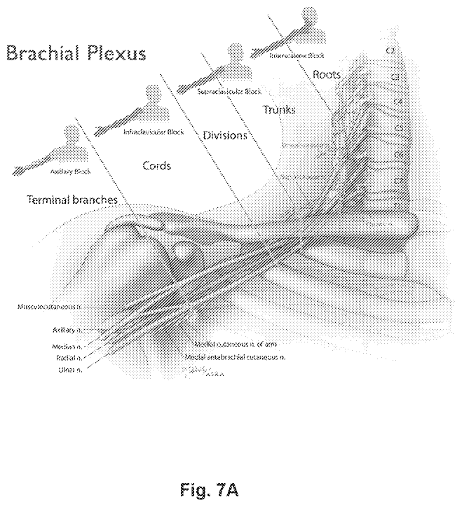

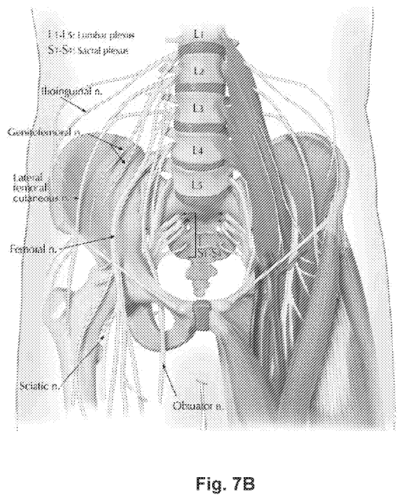

FIG. 7A is a representation of the brachial plexus nerve system which is made up of roots, trunks, divisions, cords and terminal branches.

FIG. 7B is a representation of the lumbar plexus and the sacral plexus nerve systems including the femoral nerves, obturator nerve and the lateral femoral cutaneous nerve.

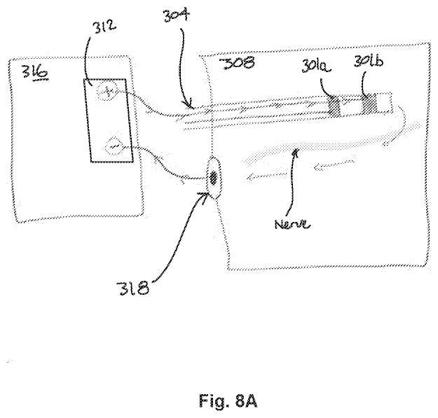

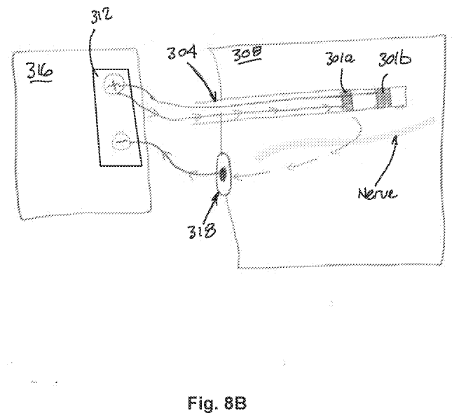

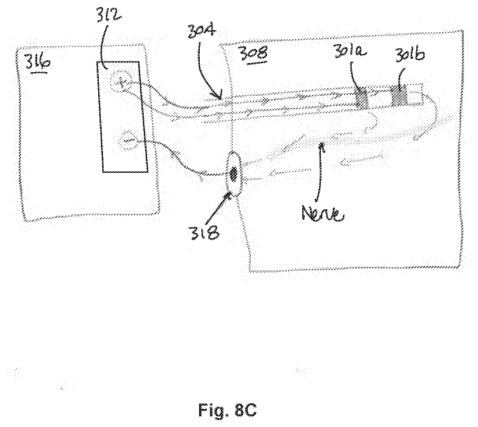

FIG. 8A shows current flowing (see arrows) from the electrical pulse generator 316 to the distal electrode on the catheter; the current travels in the tissue and returns to the electrical pulse generator via skin patch (see also, e.g., FIGS. 3A-B).

FIG. 8B shows current flowing (see arrows) from the external pulse generator 316 to the proximal electrode on the catheter; the current flows through the tissue and returns back to the external pulse generator via a skin patch (see also, e.g., FIGS. 3A-B).

FIG. 8C shows current of the same polarity flowing (see arrows) to the electrode pair on the catheter and the current returns back to the external pulse generator via skin patch (see also, e.g., FIGS. 3A-B).

### Current Flow and Placement

In one embodiment, the current flows from the electrical pulse generator to the distal electrode on the catheter (see FIG. 8A); the current travels in the tissue and returns to the electrical pulse generator via a skin patch. In this configuration, the distal electrode functions as a unipolar electrode. The proximal electrode on the catheter is “electrically silent.”

In another embodiment, the current flows from the external pulse generator to the proximal electrode on the catheter (see FIG. 8B); the current flows through the tissue and returns back to the external pulse generator via skin patch. In this iteration, the proximal electrode functions as a unipolar electrode. The distal electrode on the catheter is “electrically silent.”

In another embodiment, the current of the same polarity flows to the electrode pair on the catheter (see FIG. 8C) and the current returns back to the external pulse generator via skin patch.

In this iteration, both the electrodes are electrically active and of the same polarity.

The electrical pulse generator, in some embodiments, is designed to be used with electrodes on needle and/or catheters that function as unipolar electrode.

The electrical pulse generator can also be designed to support current flow from one electrode to the second electrode of an electrode pair on the catheter.

### Analgesic/Anesthetic—Sodium Channel Blockers

In some embodiments, the present disclosure is directed to method for treating pain comprising the administration of a therapeutic agent and neuromodulation. In some embodiments, the present disclosure is directed to method for treating pain comprising the administration of a sodium channel blocker and neuromodulation. In some embodiments, the sodium channel blocker and neuromodulation are provided by one or more catheters. When only one catheter is used, the sodium channel blocker medication can be injected through the catheter first and when the effects of the medication has worn off, neuromodulation can be started. In some embodiments, the sodium channel blocker is administered into a nerve trunk by a first catheter and the neuromodulation is provided by a second catheter to the same or different nerve.

In some embodiments, the therapeutic agent is a sodium channel blocker. As used herein, “sodium channel blockers” refers to a class of drugs that act by inhibition of sodium influx through sodium-potassium pump on cell membranes. Voltage-gated sodium channels (VGSCs) play a fundamental role in controlling cellular excitability and their abnormal activity is related to several pathological processes, including cardiac arrhythmias, epilepsy, neurodegenerative diseases, spasticity, chronic and neuropathic pain. Blockade of sodium channels slows the rate and amplitude of initial rapid depolarization, reduces cell excitability, and reduces conduction velocity. Sodium channel blockade can also stop transmission of impulse (depolarization) in a nerve trunk resulting in nerve block and is the basis for surgical anesthesia and analgesia.

There are three subgroups that, in addition to affecting phase 0 of the action potentials, sodium channel blockers may also alter the action potential duration (APD) and effective refractory period (ERP). Because some sodium channel blockers increase the ERP (class 1-A), while others decrease it (class 1-B) or have no effect on it (class 1-C), the Vaughan-Williams classification recognizes these differences as subclasses.

In some embodiments, the sodium channel blocker medications used for injecting through the catheter to block the sodium channel blocker are local anesthetic solutions. These medications classified in to two different chemical groups based on whether they have an Amide or Ester bond present in their chemical structure. In some embodiments, local anesthetics for administration from the amide group include Bupivacaine, Ropivacaine, Lidocaine, Mepivacaine etc. Esters group local anesthetics include tetracaine, chloroprocaine etc.

In some embodiments, the local anesthetics are used clinically in a variety of clinically appropriate concentrations. For example, in some embodiments, Bupivacaine can be used in following concentrations: 0.0125%, 0.25%, 0.5%. 0.75% and/or 1.3%. In some embodiments, Lidocaine can be used in the following concentrations: 0.5%, 1%, 1.5%, 2%, 4% and 5%.

The local anesthetics solutions of both groups can be used for injection through the catheters to block the sodium channel in a various concentrations.

In some embodiments, a local anesthetic may be prepared with another substance for slow of gradual release of the local anesthetic solution. In some embodiments, the local anesthetic is Exparel which is bupivacaine molecules enclosed in liposomes (depofoam) that are released gradually and prolong the half-life of bupivacaine. In some embodiments, the local anesthetic is bound in a thermal setting gel. All approved formulations can be used for injection in through the medication catheter of the present disclosure. In some embodiments, the sodium channel blocker is POSIMIR. In some embodiments, the sodium channel blocker is EXPAREL.

In some embodiments, the local anesthetics are combined with adjuvants to prolong the duration of sodium channel blockade. In some embodiments, the adjuvant is Epinephrine, dexamethasone, dexmetomidine, clonidine, fentanyl, neostigmine etc. If more adjuvants are studied and used for perineural injections and are safe and efficacious, they can used in the medication catheter of the present disclosure.

In some embodiments, drugs that may be used both along and in combination with for sodium channel blockade are NaV blockers, TRPV-1 agonists etc.

### Pain Treatment

In some embodiments, the methods of the present disclosure are directed to treating acute pain. In some embodiments, the methods of the present disclosure are directed to treating chronic pain. In some embodiments, the method of the present disclosure is directed to either treating acute pain or managing chronic pain over a period of time. In some embodiments, the method of the present disclosure is directed to treating acute pain and managing chronic pain over a period of time. In some embodiments, the method of the present disclosure is directed to treating acute pain followed by managing chronic or persistent pain over a period of time.

In some embodiments, the present disclosure is directed to a method of treating severe pain comprising administering a sodium channel blocker via a first catheter and a neuromodulation impulse via a second catheter, wherein the sodium channel blocker is administered further away from the brain, brain stem, and/or spinal cord until the severe acute pain subsides followed by continual administration of neuromodulation impulses. In some embodiments, the present disclosure is directed to a method of treating severe pain comprising simultaneously administering a sodium channel blocker via a first catheter and a neuromodulation impulse via a second catheter, wherein the sodium channel blocker is administered further away from the brain or brain stem until the severe acute pain subsides followed by continual administration of neuromodulation impulses. In some embodiments, only one catheter is used, initially to administer the sodium channel blocker medication to a target nerve and when the effects of the sodium channel medication wear off, the same catheter is used to administer electrical pulse signal for neuromodulation to the same nerve.

In some embodiments, the methods herein directed to treating a wide variety of pain types and locations. In some embodiments, the methods herein are for the treatment of orthopedic pain. In some embodiments, the methods herein are directed to the treatment of upper extremity (e.g. hand, wrist, elbow, shoulder) and lower extremity (e.g. knee, hip, foot and ankle pain). In some embodiments, the methods herein are for the treatment of the trunk (e.g. chest wall, breast, abdominal wall, back) with erector spinae plane block or paravertebral block or Quadratus lumborum block. In some embodiments, the methods here are for the treatment of chronic pain. In some embodiments, the methods herein are for the treatment of shoulder pain, acute post-operative pain, acute post-operative shoulder pain, facet joint pain, rotator cuff pain, chronic regional pain syndrome, inguinal hernia pain, and/or phantom limb pain.

In some embodiments, the methods of the present disclosure are directed to the treatment of a location on a nerve associated with pain. In some embodiments, the targeted location on a pain-associated nerve is closer to the brain than the catheters. In some embodiments, the targeted location on a pain-associated nerve is between the catheters and the brain. In some embodiments, the targeted location on a pain-associated nerve is at the location of both catheters. In some embodiments, the targeted location on a pain-associated nerve is upstream compared to both catheters. In some embodiments, both catheters are upstream compared to the targeted location on a pain-associated nerve. In some embodiments, the targeted location on a pain-associated nerve is downstream compared to both catheters. In some embodiments, both catheters are downstream compared to the targeted location on a pain-associated nerve.

In some embodiments, the method of the present disclosures are directed to the treatment of pain associated with one or more nerve selected from the group consisting of Abdominal aortic plexus, Abducens nerves, Accessory nerve, Accessory obturator nerve, Alderman's nerve, Anococcygeal nerve, Ansa cervicalis, Anterior interosseous nerve, Anterior superior alveolar nerve, Auerbach's plexus, Auriculotemporal nerve, Axillary nerve, Brachial plexus, Buccal branch of the facial nerve, Buccal nerve, Cardiac plexus, Cavernous nerves, Cavernous plexus, Celiac ganglia, Cervical branch of the facial nerve, Cervical plexus, Chorda tympani, Ciliary ganglion, Coccygeal nerve, Cochlear nerve, Common fibular nerve, Common palmar digital nerves of median nerve, Deep branch of the radial nerve, Deep fibular nerve, Deep petrosal nerve, Deep temporal nerves, Diagonal band of Broca, Digastric branch of facial nerve, Dorsal branch of ulnar nerve, Dorsal nerve of clitoris, Dorsal nerve of the penis, Dorsal scapular nerve, Esophageal plexus, Ethmoidal nerves, External laryngeal nerve, External nasal nerve, Facial nerve, Femoral nerve, Frontal nerve, Gastric plexuses, Geniculate ganglion, Genital branch of genitofemoral nerve, Genitofemoral nerve, Glossopharyngeal nerve, Greater auricular nerve, Greater occipital nerve, Greater petrosal nerve, Hepatic plexus, Hypoglossal nerve, Iliohypogastric nerve, Ilioinguinal nerve, Inferior alveolar nerve, Inferior anal nerves, Inferior cardiac nerve, Inferior cervical ganglion, Inferior gluteal nerve, Inferior hypogastric plexus, inferior mesenteric plexus, Inferior palpebral nerve, infraorbital nerve, infraorbital plexus, Infratrochlear nerve, Intercostal nerves, Intercostobrachial nerve, Intermediate cutaneous nerve, Internal carotid plexus, Internal laryngeal nerve, Interneuron, Jugular ganglion, Lacrimal nerve, Lateral cord, Lateral cutaneous nerve of forearm, Lateral cutaneous nerve of thigh, Lateral pectoral nerve, Lateral plantar nerve, Lateral pterygoid nerve, Lesser occipital nerve, Lingual nerve, Long ciliary nerves, Long root of the ciliary ganglion, Long thoracic nerve, Lower subscapular nerve, Lumbar nerves, Lumbar plexus, Lumbar splanchnic nerves, Lumboinguinal nerve, Lumbosacral plexus, Lumbosacral trunk, Mandibular nerve, Marginal mandibular branch of facial nerve, Masseteric nerve, Maxillary nerve, Medial cord, Medial cutaneous nerve of arm, Medial cutaneous nerve of forearm, Medial cutaneous nerve, Medial pectoral nerve, Medial plantar nerve, Medial pterygoid nerve, Median nerve, Meissner's plexus, Mental nerve, Middle cardiac nerve, Middle cervical ganglion, Middle meningeal nerve, Motor nerve, Muscular branches of the radial nerve, Musculocutaneous nerve, Mylohyoid nerve, Nasociliary nerve, Nasopalatine nerve, Nerve of pterygoid canal, Nerve to obturator internus, Nerve to quadratus femoris, Nerve to the Piriformis, Nerve to the stapedius, Nerve to the subclavius, Nervus intermedius, Nervus spinosus, Nodose ganglion, Obturator nerve, Oculomotor nerve, Olfactory nerve, Ophthalmic nerve, Optic nerve, Otic ganglion, Ovarian plexus, Palatine nerves, Palmar branch of the median nerve, Palmar branch of ulnar nerve, Pancreatic plexus, Patellar plexus, Pelvic splanchnic nerves, Perforating cutaneous nerve, Perineal branches of posterior femoral cutaneous nerve, Perineal nerve, Petrous ganglion, Pharyngeal branch of vagus nerve, Pharyngeal branches of glossopharyngeal nerve, Pharyngeal nerve, Pharyngeal plexus, Phrenic nerve, Phrenic plexus, Posterior auricular nerve, Posterior branch of spinal nerve, Posterior cord, Posterior cutaneous nerve of arm, Posterior cutaneous nerve of forearm, Posterior cutaneous nerve of thigh, Posterior scrotal nerves, Posterior superior alveolar nerve, Proper palmar digital nerves of median nerve, Prostatic plexus (nervous), Pterygopalatine ganglion, Pudendal nerve, Pudendal plexus, Pulmonary branches of vagus nerve, Radial nerve, Recurrent laryngeal nerve, Renal plexus, Sacral plexus, Sacral splanchnic nerves, Saphenous nerve, Sciatic nerve, Semilunar ganglion, Sensory nerve, Short ciliary nerves, Sphenopalatine nerves, Splenic plexus, Stylohyoid branch of facial nerve, Subcostal nerve, Submandibular ganglion, Suboccipital nerve, Superficial branch of the radial nerve, Superficial fibular nerve, Superior cardiac nerve, Superior cervical ganglion, Superior ganglion of glossopharyngeal nerve, Superior ganglion of vagus nerve, Superior gluteal nerve, Superior hypogastric plexus, Superior labial nerve, Superior laryngeal nerve, Superior lateral cutaneous nerve of arm, Superior mesenteric plexus, Superior rectal plexus, Supraclavicular nerves, Supraorbital nerve, Suprarenal plexus, Suprascapular nerve, Supratrochlear nerve, Sural nerve, Sympathetic trunk, Temporal branches of the facial nerve, Third occipital nerve, Thoracic aortic plexus, Thoracic splanchnic nerves, Thoraco-abdominal nerves, Thoracodorsal nerve, Tibial nerve, Transverse cervical nerve, Trigeminal nerve, Trochlear nerve, Tympanic nerve, Ulnar nerve, Upper subscapular nerve, Uterovaginal plexus, Vagus nerve, Ventral ramus, Vesical nervous plexus, Vestibular nerve, Vestibulocochlear nerve, Zygomatic branches of facial nerve, Zygomatic nerve, Zygomaticofacial nerve, and the Zygomaticotemporal nerve.

In some embodiments, the target nerve is the medial branch block of spinal nerves. In some embodiments, the target nerve is the medial branch block of spinal nerves for treating back pain originating from the facet joint in the spine. In some embodiments, the target nerve is the medial branch block of spinal nerves for treating chronic back pain originating from the facet joint in the spine.

In some embodiments, the target nerve is the suprascapular nerve. In some embodiments, the target nerve is the axillary nerve. In some embodiments, the target nerve is the suprascapular nerve for treating shoulder pain. In some embodiments, the target nerve is the axillary nerve for treating shoulder pain. In some embodiments, the target nerve is the suprascapular nerve for treating chronic shoulder pain. In some embodiments, the target nerve is the axillary nerve for treating chronic shoulder pain.

In some embodiments, the target nerve is the femoral nerve, the sciatic nerve, the obturator nerve or their branches. In some embodiments, the target nerve is the femoral nerve the sciatic nerve, the obturator nerve or its branches for knee pain. In some embodiments, the target nerve is the femoral nerve the sciatic nerve, the obturator nerve or their branches for post-surgical knee pain.

In some embodiments, multiple nerves are targeted. In some embodiments, the one nerve is targeted for neuromodulation and a different nerve is targeted for administration of a therapeutic agent, optionally a sodium channel blocker as discussed herein. In some embodiments, more than one nerve is targeted for administration of a therapeutic agent. In some embodiments, the femoral nerve is targeted for neuromodulation and the saphenous nerve and nerve to vastus medialis is targeted for administration of a therapeutic agent, optionally a sodium channel blocker.

Therefore, embodiments of the present disclosure utilize both modalities of molecular block with sodium channel blocker (nerve block), and electrical stimulation (neuromodulation; synaptic gate block) to control the entire episode of pain. The sodium channel blockade controls the early severe post-surgical pain, and as the effects of the sodium channel blockade dissipate, neuromodulation is used to control the moderate-mild pain that ensues. In case there is transient breakthrough pain, the sodium channel blockader can be used to control the analgesic gap that may occur.

In some embodiments, the location where the sodium channel blockade and electrical stimulation is applied to a nerve trunk is important for system to function efficaciously. To this end, the electrical stimulation is applied on the nerve trunk closer to the spinal cord relative to the location of where then molecular blockade sodium channel is applied on the nerve. In some embodiments, the electrical current pulse travels to the spinal cord uninterrupted to block the synaptic gate in the dorsal horn.

Accordingly, embodiments of the present disclosure are configured to control the entire episode of post-surgical pain. The following are a few embodiments regarding placement of the drug catheter and neuromodulation in acute post-surgical pain in the upper extremity and lower extremity.

### Catheter Placement

### The Upper Extremity

The upper extremity is innervated by ventral rami of the spinal nerves that derive from C-5 to T-1 levels (nerve roots). These nerves combine and divide and re-combine to form the brachial plexus or network of nerves that ultimately give individual peripheral nerves that innervate the entire upper extremity (see Figure of Brachial plexus). The brachial plexus made up roots, trunks, divisions, cords and terminal branches. The brachial plexus may be blocked at various location along its path. The blocks are named by the location of where they are performed. In some embodiments, the brachial plexus may be performed at the level Roots/Trunks (Interscalene block), Division/Trunks (Supra-clavicular block), Cords (Infra-clavicular) and/or Terminal Branches (Axillary block). The interscalene block is closer to the spinal cord compared to the supra-clavicular block. The supra-clavicular block is closer to the spinal cord compared to infra-clavicular block. The infra-clavicular block is closer to the spinal cord compared to the Axillary block.

In some embodiments, to block the anatomic structures of the upper extremity with electrical stimulation (neuromodulation) of nerves (synaptic gate block) and nerve block (molecular gate-sodium channel block) with local anesthetic, the following tables shows the location of where the electrical stimulation and nerve block may be performed according to some embodiments.

Accordingly, the following are example locations, according to some embodiments, of the electrical stimulation catheter and the medication catheter for upper extremity site pain.

| | Electrical Stimulation | Medication Catheter |
|---|---|---|
| | Catheter | (Molecular Gate Sodium |
| Site of Surgery | (Synaptic Gate block) | Channel Block) |
| Shoulder | Interscalene block | Superior Trunk block |
| | | Alternative: Suprascapular |
| | | n block |
| Arm | Interscalene block | Supraclavicular block |
| Elbow | Supraclavicular block | Infraclavicular block |
| | Alternative: | Alternative: Axillary block |
| | Supraclavicular block | |
| Forearm | Supraclavicular block | Infraclavicular block, |
| | | Alternative: Axillary block |
| Hand/Wrist | Supraclavicular block | Infraclavicular block, |
| | | Alternative: Axillary block |
| | | or Median nerve block and/ |
| | | or Ulnar nerve block and/or |
| | | Radial Nerve Block |

### Lower Extremity

The lower extremity is innervated by nerves that are formed in the Lumbar plexus and the Sacral Plexus. The Lumbar plexus is formed by the ventral rami of L-1 to L-3 nerve roots. The plexus is forms within the belly of psoas muscle. The three main branches of the lumbar plexus that innervate the front and inner part of thigh and front of knee are; 1. Femoral nerves 2. Obturator nerve 3. Lateral Femoral Cutaneous Nerve. The Sacral plexus is formed by ventral rami of L-4 to S-3 nerve roots. Sciatic nerve is the main nerve that supplies the posterior part of the thigh, back part of knee, leg, ankle and foot.

In some embodiments, to block the anatomic structures of the lower extremity with electrical stimulation (neuromodulation) of nerves (synaptic gate) and nerve block (molecular gate-sodium channel) with local anesthetic, the following tables shows the location of where the electrical stimulation and nerve block may be performed, according to some embodiments.

| | Electrical Stimulation | |
|---|---|---|
| | Catheter | Medication catheter (Molecular |
| Site of Surgery | (Synaptic Gate block) | Gate Sodium Channel Block) |
| Hip | Lumbar plexus (psoas | PENG block and Lateral |
| | compartment) block | Femoral Cutaneous Nerve block |
| Thigh (Front) | Lumbar plexus (psoas | Femoral Nerve block |
| | compartment) block | |
| Thigh (back) | Parascacral sciatic | Sciatic nerve block (Gluteal |
| | nerve block | or infra-gluteal approach) |
| Knee (Front) | Femoral nerve block | Adductor Canal Block |
| Knee (back) | Sciatic nerve block | iPACK Block |
| | (gluteal or infra- | Sciatic (Popliteal) Nerve block |
| | gluteal approach) | |
| Leg, Foot & | Sciatic nerve block | Sciatic (Popliteal) Nerve block |
| Ankle | (gluteal or infra- | Alternative: Ankle block |
| | gluteal approach) | (only for foot and ankle pain) |

The first location of the first catheter and second location of the second catheter are on the same nerve trunk. The first location of the first catheter is further away from the brain than the second location of the second catheter. In some embodiments, the second location of the second catheter is located between the brain and the first location of the first catheter. In some embodiments, the first catheter at the first location supplies a therapeutic amount of a sodium channel blocker or pharmaceutical composition thereof. In some embodiments, the second catheter at the second location supplies an electrode for an electrical impulse. In some embodiments, the catheter including at least one electrode is located closer to the brain, dorsal ganglion, and/or spinal cord compared to the catheter configured to deliver an amount of substance to and/or around the nerve trunk.

In some embodiments, there is a distance between the first location of the first catheter and the second location of the second catheter. In some embodiments, the distance between the first location of the first catheter and the second location of the second catheter is 30 cm or less. In some embodiments, the distance between the first location of the first catheter and the second location of the second catheter is 29 cm or less. In some embodiments, the distance between the first location of the first catheter and the second location of the second catheter is 28 cm or less. In some embodiments, the distance between the first location of the first catheter and the second location of the second catheter is 27 cm or less. In some embodiments, the distance between the first location of the first catheter and the second location of the second catheter is 26 cm or less. In some embodiments, the distance between the first location of the first catheter and the second location of the second catheter is 25 cm or less. In some embodiments, the distance between the first location of the first catheter and the second location of the second catheter is 24 cm or less. In some embodiments, the distance between the first location of the first catheter and the second location of the second catheter is 23 cm or less. In some embodiments, the distance between the first location of the first catheter and the second location of the second catheter is 30 cm or less. In some embodiments, the distance between the first location of the first catheter and the second location of the second catheter is 22 cm or less. In some embodiments, the distance between the first location of the first catheter and the second location of the second catheter is 21 cm or less. In some embodiments, the distance between the first location of the first catheter and the second location of the second catheter is 20 cm or less. In some embodiments, the distance between the first location of the first catheter and the second location of the second catheter is 19 cm or less. In some embodiments, the distance between the first location of the first catheter and the second location of the second catheter is 18 cm or less. In some embodiments, the distance between the first location of the first catheter and the second location of the second catheter is 17 cm or less. In some embodiments, the distance between the first location of the first catheter and the second location of the second catheter is 16 cm or less. In some embodiments, the distance between the first location of the first catheter and the second location of the second catheter is 15 cm or less. In some embodiments, the distance between the first location of the first catheter and the second location of the second catheter is 14 cm or less. In some embodiments, the distance between the first location of the first catheter and the second location of the second catheter is 13 cm or less. In some embodiments, the distance between the first location of the first catheter and the second location of the second catheter is 12 cm or less. In some embodiments, the distance between the first location of the first catheter and the second location of the second catheter is less than 11 cm. In some embodiments, the distance between the first location of the first catheter and the second location of the second catheter is less than 10 cm. In some embodiments, the distance between the first location of the first catheter and the second location of the second catheter is less than 9 cm. In some embodiments, the distance between the first location of the first catheter and the second location of the second catheter is less than 8 cm. In some embodiments, the distance between the first location of the first catheter and the second location of the second catheter is less than 7 cm. In some embodiments, the distance between the first location of the first catheter and the second location of the second catheter is less than 6 cm. In some embodiments, the distance between the first location of the first catheter and the second location of the second catheter is less than 5 cm. In some embodiments, the distance between the first location of the first catheter and the second location of the second catheter is less than 4 cm. In some embodiments, the distance between the first location of the first catheter and the second location of the second catheter is less than 3 cm. In some embodiments, the distance between the first location of the first catheter and the second location of the second catheter is less than 2 cm. In some embodiments, the distance between the first location of the first catheter and the second location of the second catheter is less than 1 cm.

From about 1 cm to about 2 cm, from about 1 cm to about 3 cm, from about 1 cm to about 4 cm, from about 1 cm to about 5 cm, from about 1 cm to about 6 cm, from about 1 cm to about 7 cm, from about 1 cm to about 8 cm, from about 1 cm to about 9 cm, from about 1 cm to about 10 cm, from about 1 cm to about 11 cm, from about 1 cm to about 12 cm; from about 2 cm to about 3 cm, from about 2 cm to about 4 cm, from about 2 cm to about 5 cm, from about 2 cm to about 6 cm, from about 2 cm to about 7 cm, from about 2 cm to about 8 cm, from about 2 cm to about 9 cm, from about 2 cm to about 10 cm, from about 2 cm to about 11 cm, from about 2 cm to about 12 cm; from about 3 cm to about 4 cm, from about 3 cm to about 5 cm, from about 3 cm to about 6 cm, from about 3 cm to about 7 cm, from about 3 cm to about 8 cm, from about 3 cm to about 9 cm, from about 3 cm to about 10 cm, from about 3 cm to about 11 cm, from about 3 cm to about 12 cm; from about 4 cm to about 5 cm, from about 4 cm to about 6 cm, from about 4 cm to about 7 cm, from about 4 cm to about 8 cm, from about 4 cm to about 9 cm, from about 4 cm to about 10 cm, from about 4 cm to about 11 cm, from about 4 cm to about 12 cm; from about 5 cm to about 6 cm, from about 5 cm to about 7 cm, from about 5 cm to about 8 cm, from about 5 cm to about 9 cm, from about 5 cm to about 10 cm, from about 5 cm to about 11 cm, from about 5 cm to about 12 cm; from about 6 cm to about 7 cm, from about 6 cm to about 8 cm, from about 6 cm to about 9 cm, from about 6 cm to about 10 cm, from about 6 cm to about 11 cm, from about 6 cm to about 12 cm; from about 7 cm to about 8 cm, from about 7 cm to about 9 cm, from about 7 cm to about 10 cm, from about 7 cm to about 11 cm, from about 7 cm to about 12 cm; from about 8 cm to about 9 cm, from about 8 cm to about 10 cm, from about 8 cm to about 11 cm, from about 8 cm to about 12 cm; from about 9 cm to about 10 cm, from about 9 cm to about 11 cm, from about 9 cm to about 12 cm; from about 10 cm to about 11 cm, from about 10 cm to about 12 cm; from about 11 cm to about 12 cm

### Timing

In some embodiments, the therapeutic agent (analgesic, sodium channel blocker, etc.) is administered and the neuromodulation electric impulse is administered simultaneously or essentially simultaneously. In some embodiments, the therapeutic agent (analgesic, sodium channel blocker, etc.) is administered through a first catheter at a first location and the neuromodulation electric impulse is administered via a second catheter at a second location simultaneously. In some embodiments, the therapeutic agent (analgesic, sodium channel blocker, etc.) is administered through a first catheter at a first location and the neuromodulation electric impulse is administered via a second catheter at a second location simultaneously and the first location is further from the brain stem compared to the second location.

In some embodiments, the therapeutic agent (analgesic, sodium channel blocker, etc.) is administered at an initial time point and the neuromodulation electric impulse is administered at a second time point, wherein the second time point is after the initial time point. In some embodiments, the neuromodulation impulse is administered at an initial time point and the therapeutic agent (analgesic, sodium channel blocker, etc.) is administered at a second time point, wherein the second time point is after the initial time point. The second time point is determined when the therapeutic effect of the sodium channel blocker has dissipated, and neuromodulation is initiated. The duration from the initial time point to the second time point can range from 30 minutes to 72 hours based on the half-life of the sodium channel blocking medication.

### Catheter Insertion Technique Examples

Catheter insertion for performing various methods of the disclosure can be as follows.

Electrical Stimulation Catheter. The skin is prepped with and anti-septic solution (chlorohexidine) and draped in the usual fashion. In some embodiments, an ultrasound transducer enclosed in a sterile sleeve is placed on the skin to visualize the target nerve.

In some embodiments, under live ultrasound guidance, the catheter with the needle inside is advanced towards the target nerve. In some embodiments, the proximal end of the insulated needle is connected to a nerve stimulator which is sends an electrical pulse of current (1.0 to 2.0 mAmps, 0.1 msec pulse duration at a frequency of 2-4 hertz) through the shaft of the needle to the exposed (uninsulated) needle tip which functions as a unipolar electrode. As the current exits the needle tip in the tissue, it creates an electric field at the distal end of the needle. The current exits the needle tip and travel through the tissue returns to the pulse generator via a skin electrode to complete the electrical circuit. As the catheter-needle assembly approaches the target nerve, the electrical field at the needle tip can depolarize the target nerve and the muscles innervated by nerve begin to contract and twitch. Live ultrasound guidance can also be used as additional confirmation that the needle tip is in close proximity to the nerve.

In some embodiments, the electrical pulse going through the needle is stopped and the nerve stimulator is connected to the pair of electrodes on the distal end of the catheter. The electrical pulse generator is set to deliver an electrical pulse of 100 hertz (range 100-4999 hertz) and a pulse duration of 0.03 ms (range 0.1-1.0 msec). The current intensity (mAmps) can be gradually increased till the patients feels a “buzzing” (paresthesia) sensation in the region of the body innervated by the nerve and more specifically in the site of surgical injury. In some embodiments, the current may range from 0.1 mAmps-20 mAmps (and ranges therebetween).

Adjustments in the position of the catheter can be made by moving the catheter in and out in relation to the nerve to optimize the elicitation of the paresthesia. Moreover, the setting of the electrical pulse generator can be adjusted to get obtain the best paresthesia response. The settings of the electrical pulse generator is noted (recorded) and neuromodulation of the nerve started. Alternatively, the neuromodulation is started in the recovery room before the patient is discharged from the recovery room. The needle can be withdrawn and the catheter is left in place and secured to the skin with tape.

Medication Catheter. In some embodiments, the steps for inserting the medication catheter comprises one or more of the step above regarding the electrical stimulation catheter. Accordingly, local anesthetic solution is injected through the needle in 3 mL aliquots up to 10-30 ml (depending on the nerve) close to the nerve (perineurally) to initiate a primary nerve block (sodium channel block-molecular gate). The catheter is then slid gently over the needle by 0.5 cm to 2.0 cm beyond the needle tip (catheter-over-needle technique) in the pocket of fluid created by the injection of the local anesthetic injection such that the catheter tip rests in close proximity of the nerve. The needle is then withdrawn leaving the catheter in place. The catheter is taped to the skin and its hub connected to the bolus pump.

### Example—Chronic Pain Treatment

Some embodiments of the present disclosure address chronic pain. To this end, a region of the body that is the source of chronic pain is usually innervated by multiple nerves. In treating the chronic pain effectively, it is important to identify which nerve or nerves are primarily transmitting the pain signal. To pinpoint which nerve is transmitting the pain signal a series diagnostic nerve blocks are performed in a sequential manner on nerves innervating the region of the body that is the source of pain. A nerve block is performed by injecting local anesthetic solution around the nerves and determine if the nerve block leads to pain relief. If the first nerve block does not provide pain relief, other nerves are blocked one at a time to determine which peripheral nerve is involved in the transmission of pain.

Once the involved nerve is identified, that nerve may undergo electrical stimulation (neuromodulation) or ablation (destruction) with heat (thermal) or cold (Cryo) or chemical (e.g. phenol injection). Each of these steps involve multiple visits to the physician's office and multiple procedures to identify and treat the nerves.

The catheter tip is be placed in close proximity to a target nerve (as described above). For a diagnostic test, local anesthetic solution is injected through the catheter. If the ensuing nerve block provides pain relief to the patient, with the same catheter, electrical stimulation (neuromodulation) can be provided for 30-60 days as a therapeutic modality to control the pain.

Thus, some embodiments according to the present disclosure allow the physician to perform a diagnostic block followed by therapeutic neuromodulation treatment. For the patient, a single insertion of the catheter can serve the dual purpose of diagnosing and treating the pain, thereby avoiding multiple (unpleasant) procedures and frequent visits to the physicians' office.

### Examples of Nerves for Blocking to Address Chronic Pain

### Shoulder Pain (Non-Surgical)

1. Suprascapular nerve
2. Axillary nerve in the quadrilateral space

### Back Pain

1. Medial branch block of spinal nerves from multiple level (e.g. L4-L5 or L5-S1 etc.) or bilaterally
2. Dorsal Root ganglion stimulation at multiple levels

### Phantom Pain (Lower Extremity)

1. Femoral nerve
2. Sciatic nerve
3. Obturator nerve
   Other Condition that Nay be Treated with Methods/Systems, Device of the Present Disclosure Include:
1. Complex Regional Pain Syndrome
2. Occipital Neuralgia
3. Post-herniorrhaphy pain
4. Post Thoracotomy pain
5. Post-mastectomy pain
6. Persistent pain after surgery (e.g. post total knee replacement pain or post rotator cuff repair pain in shoulder etc.)

While various inventive embodiments have been described and illustrated herein, those of ordinary skill in the art will readily envision a variety of other means, functionality, steps, and/or structures (including software code) for performing the functionality disclosed and/or obtaining the results and/or one or more of the advantages described herein, and each of such variations and/or modifications is deemed to be within the scope of the inventive embodiments described herein. More generally, those skilled in the art will readily appreciate that all parameters, and configurations described herein are meant to be exemplary and that the actual parameters, and configurations will depend upon the specific application or applications for which the inventive teachings is/are used. Those skilled in the art will recognize or be able to ascertain using no more than routine experimentation, many equivalents to the specific inventive embodiments described herein. It is therefore to be understood that the foregoing embodiments are presented by way of example only and that, within the scope of any claims supported by this disclosure and equivalents thereto, inventive embodiments may be practiced otherwise than as specifically described and claimed. Inventive embodiments of the present disclosure are also directed to each individual feature, system, apparatus, device, step, code, functionality and/or method described herein. In addition, any combination of two or more such features, systems, apparatuses, devices, steps, code, functionalities, and/or methods, if such features, systems, apparatuses, devices, steps, code, functionalities, and/or methods are not mutually inconsistent, is included within the inventive scope of the present disclosure. Further embodiments may be patentable over prior art by specifically lacking one or more features/functionality/steps (i.e., claims directed to such embodiments may include one or more negative limitations to distinguish such claims from prior art).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

What is currently claimed:

1. A pain management method comprising:
implanting a first catheter at a first location such that a distal end of the first catheter is proximate a nerve trunk of a patient;
and
implanting a second catheter at a second location such that a distal end of the second catheter is proximate the nerve trunk and spaced away from the distal end of the first catheter;
wherein:
at least one of the first catheter and the second catheter include at least one or more electrodes configured to provide a pulsed electrical current to and/or around the nerve trunk,
at least one of the first catheter and the second catheter are configured to deliver an amount of a substance to and/or around the nerve trunk,
and
the catheter including at least one or more electrodes is located closer to the brain, dorsal ganglion, and/or spinal cord compared to the catheter configured to deliver an amount of substance to and/or around the nerve trunk.

2. A pain management method comprising:
implanting a first catheter at a first location such that a distal end of the first catheter is proximate a nerve trunk of a patient;
and
implanting a second catheter at a second location such that a distal end of the second catheter is proximate the nerve trunk and spaced away from the distal end of the first catheter;
wherein:
at least one of the first catheter and the second catheter include at least one electrode configured to provide a pulsed electrical current to and/or around the nerve trunk, and
at least one of the first catheter and the second catheter are configured to deliver an amount of a substance to and/or around the nerve trunk.

3. The method of claims 1 or 2, wherein both of the first catheter and the second catheter include at least one electrode configured to provide the pulsed electrical current to and/or around the nerve trunk.

4. The method of any of claims 1-3, wherein both of the first catheter and the second catheter are configured to deliver an anesthetic to and/or around the nerve trunk.

5. The method of claim 1 or 2, wherein the first catheter includes the at least one electrode configured to provide the pulsed electrical current to and/or around the nerve trunk, and the second catheter is configured to deliver an anesthetic to and/or around the nerve trunk.

6. The method of claim 1 or 2, wherein only the first catheter includes the at least one electrode configured to provide the pulsed electrical current to and/or around the nerve trunk, and only the second catheter is configured to deliver an anesthetic to and/or around the nerve trunk.

7. The method of any of claims 1-6, wherein the at least one electrode comprises at least a pair of electrodes.

8. The method of any of claims 1-7, wherein:
at least one of the first catheter and the second catheter includes a removable needle;
at least one of the first catheter and the second catheter includes a removable needle having a lumen.

9. The method of claim 8, wherein:
the at least one electrode comprises at least a pair of electrodes;
at least one of the first catheter and the second catheter includes a removable needle having a lumen, and
one of the electrodes of the electrode pair comprises the distal end (tip) of the needle.

10. The method of any of claims 1-9, wherein the delivery of the amount of the substance is configured to close the molecular gate of the sodium channel in the axonal membrane of the nerve trunk.

11. The method of any of claims 1-10 wherein the substance comprises at least one of a sodium channel blocker and an anesthetic.

12. The method of claim 11, wherein the amount of the at least one of the sodium channel blocker and the anesthetic amount of the anesthetic is between about 0.5 and 30 ml.

13. The method of any of claims 1-12, wherein the delivery of the pulsed electrical current is configured to close the synaptic gate in the dorsal horn of the spinal cord.

14. The method of any of claims 1-13, wherein the pulsed electrical current comprises a current of between 0.1 and 20 milliamps.

15. The method of any of claims 1-14, wherein the pulsed electrical current includes pulses of between 0.1 and 1.0 millisecond.

16. The method of any of claims 1-15, wherein the second location is spaced away from the first location by between 5 and 200 mm.

17. The method of any of claims 1-16, wherein the nerve trunk contains the nerve pathway (innervation) for a body part of the patient.

18. The method of claim 17, wherein the body part is selected from the group consisting of: upper extremity including the shoulder, the arm, the elbow, the forearm, the wrist and the hand and the lower extremity including the hip, thigh, knee, leg, ankle and foot and the spine (including the cervical, thoracic, lumbar and sacral regions of the spine) and the head and neck, chest wall and abdominal wall

19. The method of any of claims 1-18, wherein the method is performed for pain control after a surgical procedure and/or persistent pain after surgical procedure and chronic pain.

20. The method of any of claims 1-19, wherein the pulsed electrical current is supplied via a pulse generator.

21. The method of any of claims 1-20, wherein the amount of substance is supplied via a pump.

22. The method of any of claims 1-21, wherein at least one of the first catheter and second catheter are implanted under ultrasound guidance or x-ray guidance.

23. The method of any of claims 1-6, 8 and 10-22, further comprising positioning a first electrically conductive skin patch on the skin of the patient and the distal tip of the needle functioning as an electrode are configured to complete an electrical circuit with a/the pulse generator and distal end of the needle

24. The method of any of claims 1-23, further comprising, delivery the substance in and/or around the nerve trunk via the at least one of the first catheter and second catheter.

25. The method of claim 24, wherein the first substance is delivered for a first time period.

26. The method of any of claims 1-23, further comprising, delivery the pulsed electrical current in and/or around the nerve trunk via the at least one of the first catheter and second catheter having the at least one electrode.

27. The method of claim 26, wherein the pulsed electrical current is delivered for second time period.

28. The method of claim 25, further comprising, delivery the pulsed electrical current in and/or around the nerve trunk via the at least one of the first catheter and second catheter having the at least one electrode.

29. The method of claim 28, wherein the pulsed electrical current is delivered for second time period.

30. The method of claim 26 or 28, wherein the pulsed electrical current is delivered after delivery of the substance by at least one of the first catheter and the second catheter.

31. The method of claim 26 or 28, wherein the pulsed electrical current is delivered a period of time after delivery of the substance by at least one of the first catheter and the second catheter.

32. The method of any of claims 1-31, wherein the catheter that is configured with the at least one electrode is positioned between 1 cm and 12 cm closer to the brain, dorsal ganglion, and/or spinal cord when compared to the catheter configured to deliver an amount of a substance to and/or around the nerve trunk.

33. The method of any of claims 1-32, wherein the catheter that is configured with the at least one electrode is positioned between 2 cm and 5 cm closer to the brain, dorsal ganglion, and/or spinal cord when compared to the catheter configured to deliver an amount of a substance to and/or around the nerve trunk.

34. The method of any of claims 1-33, wherein the substance is a sodium channel blocker.

35. The method of claim 34, wherein the sodium channel blocker bupivacaine, ropivacaine, lidocaine, mepivacaine, chloroprocaine or a salt, combination, or pharmaceutical composition thereof.

36. The method of claim 34 or 35, wherein the sodium channel blocker is bupivacaine, lidocaine, or a salt, combination, or pharmaceutical composition thereof.

37. The method of any of claim 34-36, wherein the sodium channel blocker is a lidocaine hydrochloride solution with dextrose, wherein dextrose is present in about 1%, about 2%, about 3%, about 4%, or about 5%.

38. The method of any of claim 34-37, wherein the sodium channel blocker is a 0.2%, 0.4% or 0.8% lidocaine hydrochloride solution.

39. The method of any of claim 34-36, wherein the sodium channel blocker is a bupivacaine hydrochloride solution with dextrose, wherein dextrose is present in about 1%, about 2%, about 3%, about 4%, or about 5%.

40. The method of any of claim 34-36 or 39, wherein the sodium channel blocker is a 0.125%, 0.25%, 0.5% or 0.75%, 1.3% bupivacaine hydrochloride solution.

41. A pain management system or kit comprising:
a first catheter configured for implantation at a first location such that a distal end of the first catheter is proximate a nerve trunk of a patient;
and
a second catheter configured for implantation at a second location such that a distal end of the second catheter is proximate the nerve trunk and spaced away from the distal end of the first catheter;
wherein:
at least one of the first catheter and the second catheter include at least one electrode configured to provide a pulsed electrical current to and/or around the nerve trunk, and
at least one of the first catheter and the second catheter are configured to deliver an amount of a substance to and/or around the nerve trunk.

42. The system or kit of claim 41, wherein both of the first catheter and the second catheter include at least one electrode configured to provide the pulsed electrical current to and/or around the nerve trunk.

43. The system or kit of claims 41 or 42, wherein both of the first catheter and the second catheter are configured to deliver an anesthetic to and/or around the nerve trunk.

44. The system or kit of claim 41, wherein the first catheter includes the at least one electrode configured to provide the pulsed electrical current to and/or around the nerve trunk, and the second catheter is configured to deliver the substance to and/or around the nerve trunk.

45. The system or kit of claim 41, wherein only the first catheter includes the at least one electrode configured to provide the pulsed electrical current to and/or around the nerve trunk, and only the second catheter is configured to deliver an anesthetic to and/or around the nerve trunk.

46. The system or kit of any of claims 41-45, wherein the at least one electrode comprises at least a pair of electrodes.

47. The system or kit of any of claims 41-46, wherein at least one of the first catheter and the second catheter includes a removable needle having a lumen.

48. The system or kit of claim 47, wherein:
the at least one electrode comprises at least a pair of electrodes;
at least one of the first catheter and the second catheter includes a removable needle having a lumen, and
one of the electrodes of the electrode pair comprises the distal end of the needle.

49. The system or kit of any of claims 41-48, further comprising a vial of the substance.

50. The system or kit of claim 49, wherein the substance comprises at least one of a sodium channel blocker and an anesthetic.

51. The system or kit of any of claims 41-50, further comprising a pulse generator for supplying the pulsed electrical current.

52. The system or kit of any of claims 41-51, further comprising a pump for supplying the substance to the at least one of the first catheter and second catheter.

53. The system or kit of any of claims 41-52, further comprising a garment wearable by the patient for housing at least one of a/the pulse generator and a/the pump.

54. The system or kit of any of claims 41-53, further comprising at least one of the following components:
at least one electrical cord for at least one of the first catheter and the second catheter;
at least one needle for at least one of the first catheter and the second catheter;
at least one tube-set for at least one of the first catheter and the second catheter for connection thereof with a/the pump for delivering the substance;
at least one filter configured to be in fluid communication with at least one of the first catheter and the second catheter, the filter to configured to filter the substance prior to the substance being delivered to the nerve trunk;
at least one dressing configured to retain at least one of the first catheter and the second catheter in place while implanted into tissue of the patient;
at least one electrical cord configured to communication electrical current between a/the pulse generator and at least one of the first catheter and the second catheter;
at least one EKG patch, configured to receive pulsed electrical current from the patient via the skin surface;
at least one EKG patch cord;
at least one biopatch configured to prevent infection at skin insertion site of catheter;
at least one hub; and
at least one syringe.

55. The system or kit of claim 54, wherein the kit comprises two or more of the components.

56. The system or kit of claim 54, wherein the kit comprises a majority of the components.

57. The system or kit of claim 54, wherein the kit comprises all of the components.

58. A method, system, kit or device according to any of the disclosed embodiments.

59. A method wherein only one catheter is placed next to a nerve trunk; the catheter is used to deliver medication substance around the nerve in the first time period and subsequently the same catheter is used to deliver electrical pulse in the second period after the effect of the medication has dissipated.
